(19) United States
(12) Patent Application Publication
(10) Pub. No.: US 2017/0281231 A1
(43) Pub. Date: Oct. 5, 2017

(54) UTERINE CONTRACTION DEVICE

(71) Applicant: UNIVERSITY OF UTAH RESEARCH FOUNDATION, Salt Lake City, UT (US)

(72) Inventors: John Thomas Langell, Sandy, UT (US); Paul Sanford Holman, Murray, UT (US); Zachary Warren Eyre, Provo, UT (US); Thomas Jon Goates, Murray, UT (US); Sarah Owens Broderick, Salt Lake City, UT (US); Jared Reese Pieper, Taylorsville, UT (US)

(21) Appl. No.: 15/476,367

(22) Filed: Mar. 31, 2017

Related U.S. Application Data
(60) Provisional application No. 62/317,028, filed on Apr. 1, 2016.

(51) Int. Cl. A61B 17/42

(52) U.S. Cl. CPC A61B 17/4241; A61B 2017/00561

(57) ABSTRACT

The present disclosure describes a uterine contraction device which may be utilized to treat and/or prevent postpartum hemorrhaging. The uterine contraction device includes a catheter and a pressure seal adjustably coupled to the catheter. A distal section of the catheter is configured for insertion into the uterus, and a proximal end of the catheter is configured for attachment to a vacuum device. Negative pressure generated by the vacuum device is transmitted to the distal section, while the pressure seal forms a seal at the vaginal introitus to maintain the negative pressure within the uterus to assist in contracting the uterus.

### CROSS-REFERENCE TO RELATED APPLICATIONS

This application claims priority to and the benefit of U.S. Provisional Patent Application Ser. No. 62/317,028, filed on Apr. 1, 2016 and titled “UTERINE CONTRACTION DEVICE,” the disclosure of which is incorporated herein by this reference in its entirety.

### BACKGROUND

Postpartum hemorrhaging, or excessive bleeding after giving birth, is the leading cause of delivery-related death in the world. The problem claims approximately 150,000 lives each year, and is particularly acute in developing countries, where over 90% of such deaths occur. Postpartum hemorrhaging occurs when the uterus does not sufficiently contract to normal size after birth, which is most commonly a result of uterine muscle fatigue or caesarean section surgery. Uterine contraction stops bleeding by compressing and closing local blood vessels.

Common approaches toward postpartum hemorrhaging include use of an inflatable balloon within the uterus to provide pressure. However, these balloon tamponade solutions operate to expand rather than contract the uterus and therefore work against beneficial effects of contraction. Another approach involves delivery of uteronic drugs such as oxytocin. While such drugs can provide desired effects, they may be less effective in situations of atonic fatigue. Further, these drugs may not always be available, particularly in less developed regions. Some approaches have been proposed that operate using a pressure differential, but these approaches rely on fluid inflatable balloons and other overly complex componentry.

### BRIEF SUMMARY

The present disclosure describes a uterine contraction device which may be utilized to treat and/or prevent postpartum hemorrhaging. Certain embodiments are configured for insertion into the uterus to aid in contracting the uterus to thereby treat and/or prevent postpartum hemorrhaging.

In some embodiments, the uterine contraction device includes a catheter having a proximal end, a distal end, a distal section adjacent to the distal end, and a wall defining a lumen. The catheter is configured such that when inserted within a patient, the proximal end remains outside of the patient, and the distal end and distal section extends into the uterus. At least a portion of the distal section includes a plurality of perforations extending through the catheter wall to expose the lumen of the catheter. The proximal end of the catheter is attachable to a vacuum device to enable transmission of negative pressure through the lumen to the distal section.

The uterine contraction device also includes a pressure seal coupled to the proximal section of the catheter. In some embodiments, the pressure seal has a conical or bell shape that opens toward the proximal end of the catheter. In some embodiments, the pressure seal is adjustably coupled to the proximal section of the catheter such that the pressure seal is translatable along a longitudinal axis of the proximal section of the catheter. In some embodiments, the device further includes a locking mechanism to lock a selected position of the pressure seal relative to the catheter.

Certain embodiments are directed to a method of treating postpartum hemorrhaging in a patient. In some embodiments, the method includes providing a uterine contraction device, inserting the distal section of the medical device into the patient's uterus, positioning the pressure seal at the patient's vaginal introitus, and applying vacuum pressure to aid in contracting the uterus.

Additional features and advantages will be set forth in part in the description that follows, and in part will be obvious from the description, or may be learned by practice of the embodiments disclosed herein. The objects and advantages of the embodiments disclosed herein will be realized and attained by means of the elements and combinations particularly pointed out in the appended claims. It is to be understood that both the foregoing brief summary and the following detailed description are exemplary and explanatory only and are not restrictive of the embodiments disclosed herein or as claimed.

### BRIEF DESCRIPTION OF THE DRAWINGS

In order to describe various features and concepts of the present disclosure, a more particular description of certain subject matter will be rendered by reference to specific embodiments which are illustrated in the appended drawings. Understanding that these figures depict just some example embodiments and are not to be considered to be limiting in scope, various embodiments will be described and explained with additional specificity and detail through the use of the accompanying drawings in which:

FIGS. 1 through 4 illustrate various views of an exemplary embodiment of a uterine contraction device including a catheter and a pressure seal adjustably coupled to the catheter;

FIGS. 5 through 7 illustrate various views of an alternative embodiment of a uterine contraction device;

FIG. 8 illustrates an alternative embodiment of a uterine contraction device including a distal section that curves away from the longitudinal axis of the proximal section;

FIG. 9 illustrates a cross-sectional view of a catheter having varying wall thickness and varying outer diameter;

FIGS. 10 through 12 illustrate various exemplary embodiments of locking mechanisms which may be utilized to lock the position of the pressure seal relative to the catheter; and

FIGS. 13 and 14 illustrate an exemplary use of a uterine contraction device to treat or prevent postpartum hemorrhaging.

### DETAILED DESCRIPTION

The present disclosure describes embodiments of a uterine contraction device. Embodiments described herein may be utilized to aid in contracting a patient's uterus following childbirth in order to minimize or prevent postpartum hemorrhaging. Embodiments described herein may be utilized to collapse a patient's uterus toward an anatomically normal postpartum state. Beneficially, at least some of the embodiments described herein may be utilized to provide negative pressure within the uterus without requiring fluid inflated balloons or other relatively complex inflatable componentry.

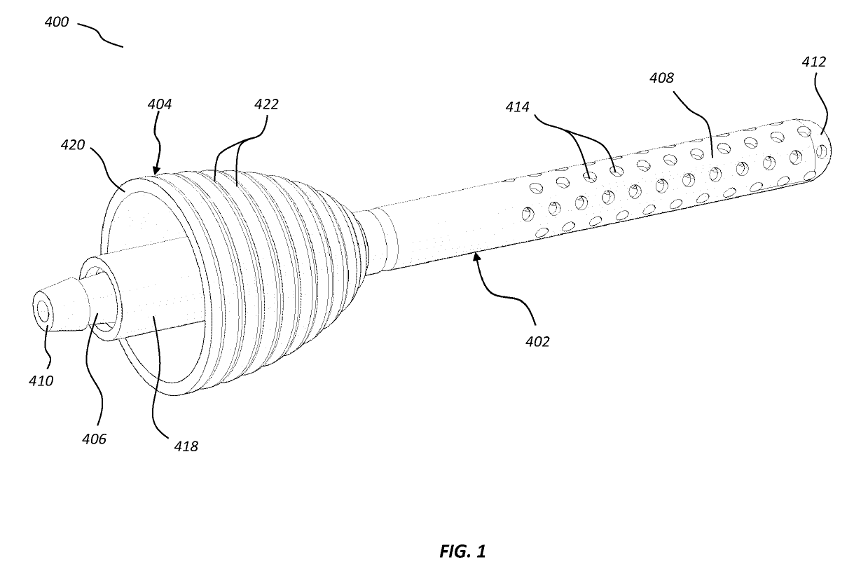

FIG. 1 illustrates an exemplary embodiment of a uterine contraction device 400. The illustrated contraction device 400 includes a catheter 402. The catheter 402 includes a stylet (referred to hereafter as proximal section 406) and a cannula (referred to hereafter as distal section 408), each respectively extending to a proximal end 410 and a distal end 412. As shown, the proximal section 406 and distal section 408 are coupled together, and are collectively referred to herein as the catheter 402. The illustrated embodiment also includes an occluder (referred to hereafter as pressure seal 404), which is coupled to the proximal section 406 of the catheter 402.

As explained in more detail below, the device 400 is configured such that, when inserted within a patient (see FIGS. 14 and 15), the proximal end 410 remains outside of the patient, and the distal end 412 and distal section 408 extend through the vaginal canal and into the uterus. When the device is inserted, the pressure seal 404 is positioned at the vaginal introitus and is configured to conform to the anatomy of the vaginal canal to seal the uterus from the external environment. Such sealing enables the application of differential (negative) pressure to the uterus, which assists in contracting the uterus and minimizing or preventing postpartum hemorrhaging.

The catheter 402 and/or pressure seal 404 may be formed from one or more suitable medical grade materials such as rubber, silicone, silastic, polyethylene, polyurethane, polycarbonate, other suitable plastics, metals, other materials suitable for temporary insertion into the body, or combinations thereof. In presently preferred embodiments, the distal section 408 has a modulus of elasticity and/or hardness (e.g., Shore A durometer) that is lower than that of the proximal section 406 and the pressure seal 404, the proximal section 406 has a modulus of elasticity and/or hardness that is greater than the pressure seal 404 and the distal section 408, and the pressure seal 404 has a modulus of elasticity and/or hardness that is intermediate to that of the proximal section 406 and distal section 408. In one example, the distal section 408 has a modulus of elasticity of about 1 to 10 MPa, the pressure seal 404 has a modulus of elasticity of about 10 to 500 MPa, and the distal section 408 has a modulus of elasticity of about 500 to 2,000 MPa.

In the illustrated embodiment, the catheter 402 is substantially straight (at least while in an unstressed/non-deformed state). In other embodiments, the proximal section and/or distal section may be curved or bent.

The distal section 408 of the illustrated device includes a plurality of perforations 414 extending to the inner lumen of the catheter 402. The perforations 414 expose the inner lumen of the catheter 402 to the external environment surrounding the distal section 408. In the illustrated embodiment, the perforations 414 are provided as rounded holes. Other embodiments may additionally or alternatively include perforations formed as slits, fenestrations, or other shapes.

The perforations 414 may be large enough to manage blood clotting while avoiding damage to uterine tissue. For example, average perforation size may be about 1 cm or less, such as about 0.1 to about 0.75 cm, or about 0.25 to about 0.5 cm, or about 0.3 to 0.4 cm. The perforations 414 are provided with appropriate spacing so as not to compromise material strength of the distal section 408. Effective transfer of negative pressure to assist in uterine contraction is provided by perforations 414 having sizes within the foregoing ranges, with about 50 to 100, or about 60 to 80 of such perforations 414 spaced apart by about 0.5 to 2 cm, or by about 1 cm along the most distal 5 to 16 cm, or about the most distal 8 to 12 cm of the distal section 408.

The perforations 414 are generally substantially uniform in size to promote uniform contraction within the treated uterus. This design consideration may also prevent ischemia and/or further damage to uterine walls, which are already relatively thin due to stretching during pregnancy. In other embodiments, the perforations 414 may have varying sizes according to particular patient and/or application needs. For example, more distally located perforations may have a different diameter than more proximally located perforations, which may enable greater contraction at more distal or more proximal areas of the uterus if such a situation is warranted by particular patient or application needs. Additionally, or alternatively, the concentration of perforations per unit of catheter surface area may be varied. For example, relatively more perforations 414 may be included at targeted portions of the distal section, which likewise may enable greater contraction at coinciding areas of the uterus.

The perforations 414 are disposed across a sufficient length of the distal section 408 to provide sufficient negative pressure and associated contraction. Preferably, the perforations 414 are disposed such that when the device 400 is inserted within a patient, the perforations 414 are disposed within a large portion of the length of the catheter within the uterus, but are not located within the vaginal canal, which could damage vaginal wall tissue.

In the illustrated embodiment, the distal end 412 of the catheter 402 also includes perforations (e.g., arranged in a circular pattern or other pattern). This can beneficially provide negative pressure at the very distal tip of the catheter 402 so that uterine contraction may be more uniformly provided when the device is operated.

As shown, the distal end 412 of the illustrated embodiment has a rounded shape. The rounded shape can beneficially prevent penetration of the uterine wall or other injury to uterine tissue when the device 400 is inserted. The modulus of elasticity of the distal section 408 is preferably high enough to provide structural rigidity to prevent excessive collapsing while under vacuum pressure, while also being low enough to provide a degree of flexibility to allow for bending in any direction necessary for proper insertion into the patient.

The proximal end 410 of the catheter 402 is attachable to a vacuum device to enable the transmission of negative pressure through the lumen of the catheter 402 to the perforations 414 of the distal section 408. The device 400 thereby enables the delivery of negative pressure to the uterus when properly inserted within a patient. The proximal end 410 may be configured as a barbed fitting, luer lock, press fitting, or other suitable structure allowing attachment to a vacuum device and/or vacuum tubing.

The illustrated pressure seal 404 has a conical, cup-like, or bell-like shape (e.g., a circular paraboloid) that opens toward the proximal end 410 of the device. The pressure seal 404 includes an outer portion 420 and an inner portion or conduit 418 at least partially disposed within the interior of the outer portion 420. As shown, the conduit 418 is positioned around the catheter 402 to join the pressure seal 404 to the catheter 402. The conduit 418 and the outer portion 420 define a hollow space within the interior of the pressure seal 404. The hollow space allows the outer portion 420 of the pressure seal 404 to radially flex inwardly so as to conform to a particular patient's vaginal anatomy. The pressure seal 404 may be sized according to typical patient anatomy, or may be sized according to a particular targeted patient's anatomy. In some embodiments, the pressure seal may have a diameter (at its largest proximal section) of about 4 to 8 cm, with the conduit 418 having an outer diameter of about 1.5 to 4 cm. The length of the pressure seal 404 may be about 4 to 8 cm, though the conduit 418 may extend further than the outer portion 420 to provide an additional 1 to 4 cm of length.

In the illustrated embodiment, the conduit 418 extends a distance proximally beyond the outer portion 420 of the pressure seal 404. This configuration can provide adequate structure for aligning the pressure seal 404 upon the catheter 402. In other embodiments, the conduit 418 does not extend proximally beyond the outer portion 420.

The shape of the pressure seal 404 enables the formation of an effective seal between the uterus and the external environment. By having a progressively wider diameter in the proximal direction, the shape of the pressure seal 404 effectively conforms to the anatomy of the vaginal canal. Additionally, the shape of the pressure seal 404, with a progressively larger diameter toward the proximal direction, may function to automatically tighten the seal as the device is inserted and/or as vacuum pressure is applied to the uterus.

In contrast, a sphere or other non-conical, non-bell-like shape would not conform as well to the surrounding anatomy and would therefore fail to form as effective of a seal. The shape of the illustrated pressure seal 404 also allows a doctor, nurse, or other caretaker utilizing the device 400 to easily visually inspect the seal and visually determine that a proper seal has been formed at the vaginal introitus. Further, the shape of the pressure seal 404 may be easily gripped by a physician, nurse, or other caretaker during insertion, removal, or adjustment of the device. For example, a physician may place his/her hand within the hollow space of the pressure seal and then grip the pressure seal 404 by wrapping his/her fingers around the conduit 418. The physician could then readily manipulate the device 400 and/or adjust the pressure seal 404.

The pressure seal 404 is preferably formed from one or more materials that provide sufficient rigidity to form a seal when positioned at a patient's vaginal introitus, while also allowing for some flexibility and bending to conform to particular patient specific anatomy. Preferably, one or more semi-rigid materials are utilized to enable the pressure seal 404 to bend and conform to the vaginal wall without collapsing and without injuring adjacent tissue. The pressure seal 404 may have a modulus of elasticity high enough to provide structural rigidity to the pressure seal 404 so that it may resist excessive collapse and so that it can maintain a sealed position within the vaginal canal, while being low enough to allow for some flexibility to allow bending to conform to the vaginal wall without collapsing or injuring vaginal tissue and to allow pliability for simple removal. The tensile strength of the device is preferably sufficient to prevent tearing after repeated sterilization and use.

In some embodiments, the pressure seal 404 includes (on at least the outer surface) an additive and/or surface coating to decrease surface roughness and to reduce or prevent adherence to adjacent tissues, which could injure the tissues and/or make removal of the device more difficult. Suitable surface coatings include lubricant fluids or jellies (e.g., silicone-based, petroleum-based), hydrogels, polymethylsiloxane (PDMS) products, and/or other medical grade additives known in the art. Other portions of the device 400 (e.g., the distal section 408 of the catheter 402) may additionally or alternatively include such an additive and/or surface coating.

As shown, the pressure seal 404 may include one or more ridges 422 formed on its outer surface. In the illustrated embodiment, the ridges 422 are circumferentially oriented, which has shown to be effective for aiding in forming the pressure seal. In other embodiments, one or more ridges may be oriented along the longitudinal length of the pressure seal 404 or may be diagonally oriented. In some embodiments, alternative textures and/or patterns may be utilized to assist in forming a seal or providing better engagement with the vaginal canal. The ridges 422 may be substantially uniformly spaced, as shown. In other embodiments, ridges may be more spatially concentrated toward the distal end of the pressure seal, or more spatially concentrated toward the proximal end of the pressure seal.

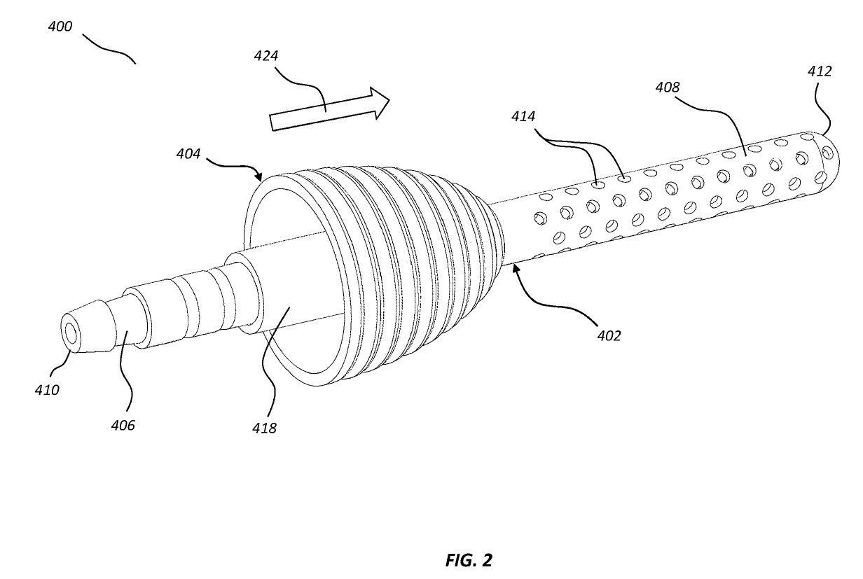

In preferred embodiments, the pressure seal 404 is adjustably coupled to the proximal section 406 of the catheter 402. In the illustrated embodiment, the conduit 418 may slide and translate upon the catheter 402, enabling a physician, nurse, or other caretaker to adjust the longitudinal position of the pressure seal 404 upon the catheter 402. For example, FIG. 1 illustrates the pressure seal 404 in a more proximal position upon the catheter 402. From the illustrated position of FIG. 1, the pressure seal 404 may be translated to a more distal position upon the catheter 402, as shown in FIG. 2 and as indicated by arrow 424. The pressure seal 404 may also be moved from the position shown in FIG. 2 to the position shown in FIG. 1 by proximally translating the pressure seal 404 upon the catheter 402. Positions in between those shown in FIGS. 1 and 2 may also be utilized by moving/translating the pressure seal 404 to the desired position.

The adjustable configuration of the pressure seal 404 beneficially allows for customized sizing of the device during insertion and use. For example, once the device has been inserted so that the distal section 408 is appropriately situated within the uterus, the pressure seal 404 may be adjusted to a position that best conforms to the patient's particular anatomy and best forms a pressure seal. Upon positioning of the pressure seal 404 at a desired position, the pressure seal position may be locked using a locking mechanism, as described in more detail below.

In contrast, a device not having a pressure seal capable of longitudinal adjustment would require that the entire device be moved just to reorient and/or properly position the seal. In many circumstances, this could lead to suboptimal positioning of the distal section in the uterus (e.g., too deep or insufficiently deep), or could lead to suboptimal formation of the vacuum seal. The illustrated embodiment instead enables desired positioning of the catheter 402 and allows for the desired position to be maintained while the pressure seal 404 is adjusted to a suitable sealing position.

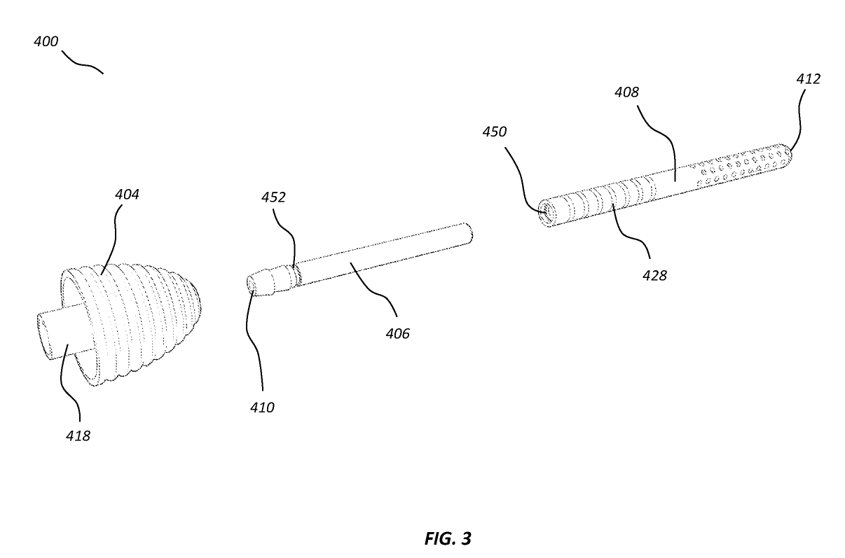

FIG. 3 illustrates an exploded view of the device 100, showing separately the distal section 408, proximal section 406, and pressure seal 404. In the illustrated embodiment, the distal section 408 includes an O-ring or reduced diameter connecting portion 450 for engaging with a corresponding groove 452 of the proximal section 406. Other embodiments may additionally or alternatively include other connection components for attaching the proximal section 406 and distal section 408. For example, the proximal section 406 may include an increased diameter portion configured to fit within an inner groove of the distal section 408, the respective sections may include threaded portions for engagement, may be adhered together, and/or may include other suitable attachment mechanisms.

In the illustrated embodiment, the proximal section 406 is configured for insertion within the distal section 408. When inserted, the proximal section 406 may extend distally within the distal section 408 a sufficient distance to provide structural support and rigidity for the portions intended to be positioned within the vaginal canal, but without extending so far distally as to interfere with the perforated region of the distal section 408. For example, the distal section 408 may have a length of about 20 to 30 cm, and the proximal section 406 may have a length of about 10 to 20 cm.

Although the catheter 402 of the illustrated embodiment is formed from two separate components, it will be understood that other embodiments may utilize a single integrated catheter, or may include more than two separate components coupled together to form the continuous catheter 402. Also, although the illustrated embodiment is configured for the proximal section 406 to be inserted within the distal section 408, alternative embodiments may be configured for insertion of the distal section 408 within the proximal section 406.

FIG. 3 also illustrates a locking mechanism configured as a series of grooves 428 for engaging with corresponding locking structures within the conduit 418 of the pressure seal 404, as explained in more detail below.

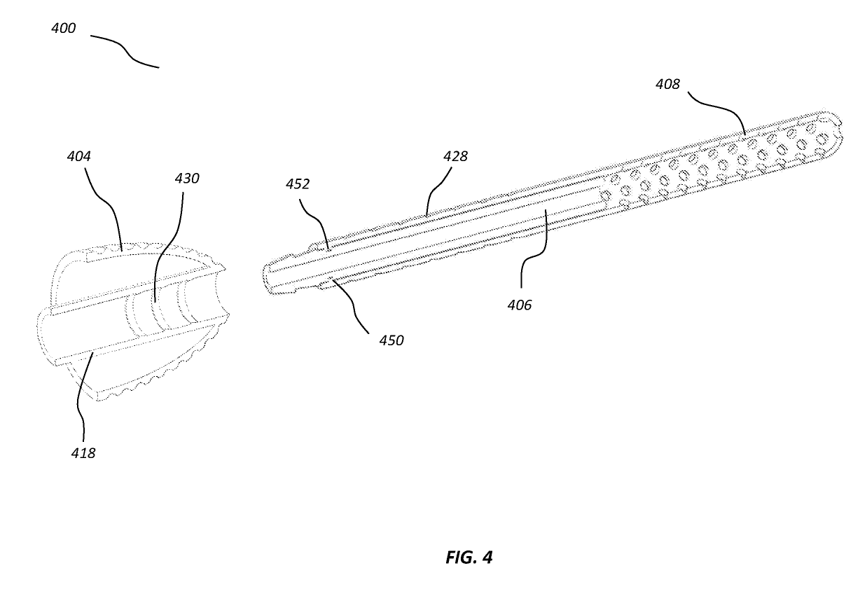

FIG. 4 illustrates the uterine contraction device 400 in cross-section, with the proximal section 406 inserted into the distal section 408 and engaged therein by the connecting portion 450 and groove 452. In the cross-sectional view, a series of extensions 430 are visible within the conduit 418 of the pressure seal 404. The extensions 430 are configured to fit and engage with the grooves 428 of the catheter 402. The grooves 428 and extensions 430 thereby enable positional locking of the pressure seal 404 upon the catheter 402, while also allowing for longitudinal adjustment of pressure seal position by moving the pressure seal 404 from one groove or set of grooves 428 to the next.

In the illustrated embodiment, the grooves 428 and extensions 430 are configured with an angled shape to more readily allow distal movement of the pressure seal 404 while generally preventing the pressure seal 404 from sliding back proximally. Typically, when a positional adjustment is required, it involves moving the pressure seal 404 further distally. During use of the device, forces will tend to push the pressure seal 404 in a proximal direction out from the vaginal canal. By preventing proximal movement but allowing distal adjustment of the pressure seal 404, the device beneficially provides for adjustment when necessary without introducing excessive and potentially detrimental freedom of movement.

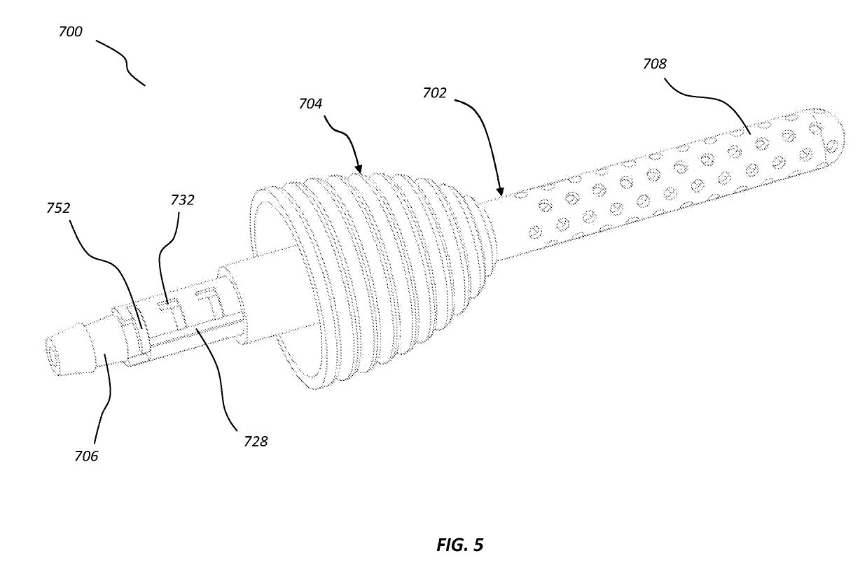

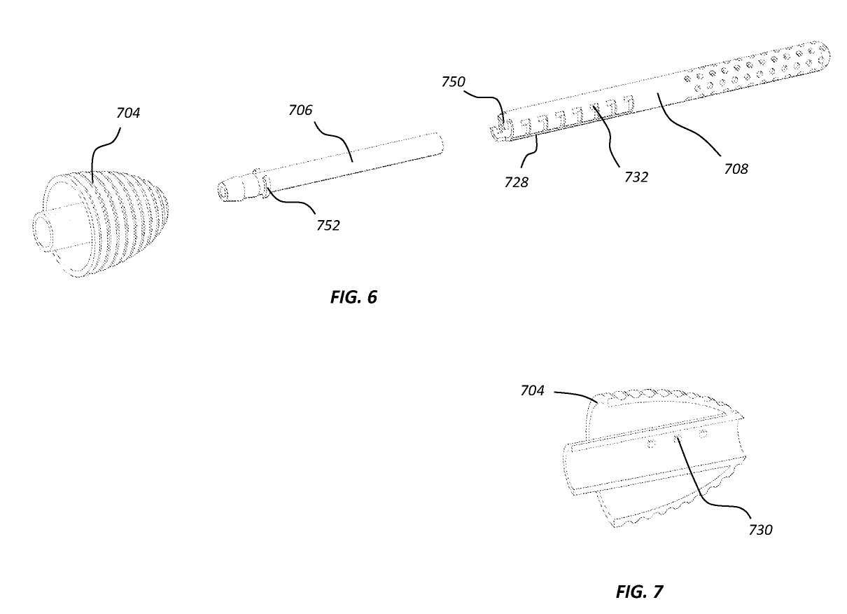

FIGS. 5 through 7 illustrate an alternative embodiment of a uterine contraction device 700. The embodiment illustrated in FIGS. 5 through 7 includes a proximal section 706 and distal section 708 that attach/detach utilizing a rotating lock mechanism. As best shown in the exploded view of FIG. 5, the proximal section 706 includes radially extending tabs 752 that correspond to receiving slots 750 of the distal section 708. Rotation of the proximal section 706 relative to the distal section 708 allows the tabs 752 to lock within the slots 750 in a manner that resists disengagement of the respective sections.

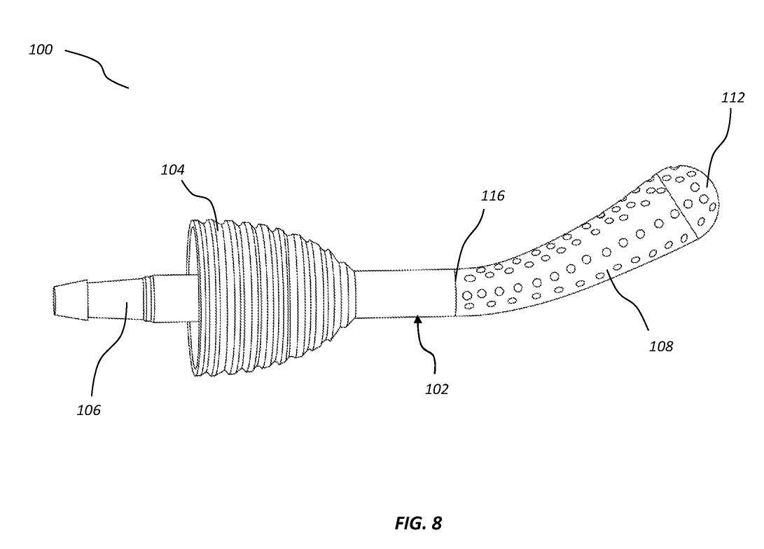

FIGS. 5 through 7 also illustrate an alternative locking mechanism. The catheter 702 includes a keyway 728 and a series of offset slots 732 connected to the keyway 728. As shown in the cross-sectional view of FIG. 7, the pressure seal 704 includes a set of keys 730 sized to fit within the keyway 728. The keys 730 allow the pressure seal 704 to be translated along the catheter 702 to a desired location. The pressure seal 704 can then be rotated enough to move the keys 730 into corresponding offset slots 732 to prevent proximal or distal movement of the pressure seal 704 until the keys 730 are rotated back into the keyway 728.

FIG. 8 illustrates another embodiment of a uterine contraction device 100 having a pressure seal 104 adjustably coupled to a curved catheter 102. In some implementations, it may be beneficial to utilize such a curved catheter 102 to allow the device to effectively conform to the contour of the female anatomy. For example, in some situations, the longest length of the uterus will not be aligned with the vaginal canal, and the uterus may extend beyond the cervix in a more anterior direction relative to the vaginal canal. The curved distal section 108 of the catheter 102 provides better conformity to this anatomy.

In some embodiments, the curved distal section 108 has a length of about 8 to 16 cm. The distal section 108 may curve away from the longitudinal axis of the proximal section 106 (i.e., the axis aligned with and extending through the proximal section 106) such that the distal end 112 is positioned about 2 to 10 cm away from, or about 4 to 8 cm away from the longitudinal axis of the proximal section 106. In some embodiments, a line extending from the inflection point 116 (where curvature begins) to the distal end 112 forms an angle rising from the longitudinal axis of the proximal section 106 of about 5 to 60 degrees, or about 15 to 45 degrees, or about 30 degrees.

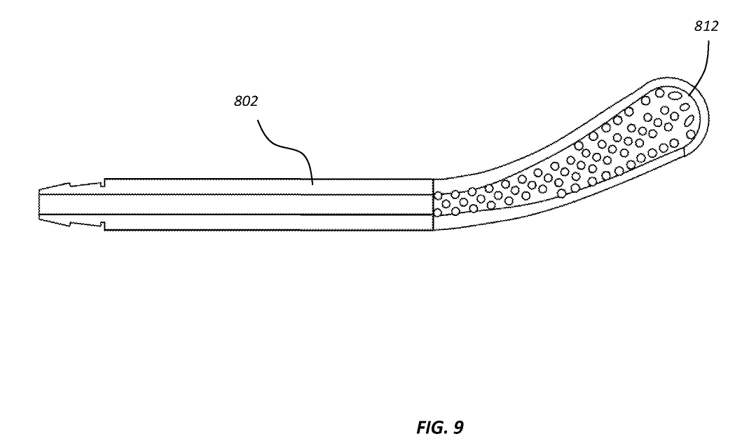

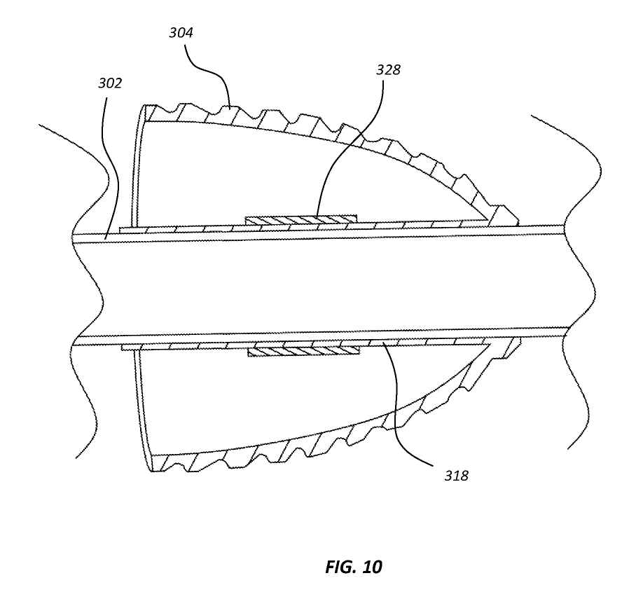

FIG. 9 illustrates a cross-sectional view of a catheter 802 with a wall that tapers in thickness to become progressively thinner toward the distal end 812. This structure can provide relatively greater pliability and compressibility to the more distal sections of the catheter 802, which are more likely to bump against and/or perforate the uterine wall during insertion and/or use. The relatively thinner walled sections thereby aid in reducing the risk of injury to and/or perforation of uterine tissues. At the same time, the thicker walled and more proximally located portions, which are less likely to directly contact the uterine walls, can provide greater structural integrity for the overall structure of the catheter 802. Although shown here as a curved catheter, the same wall profile may be utilized in non-curved catheter embodiments as well. The catheter 802 of FIG. 9 also includes a tapered outer diameter that gets larger toward the distal end 812. A larger diameter toward the distal end 812 may beneficially provide more surface area to help avoid accidental injury to tissues contacted against the distal end 812 during insertion and/or use.

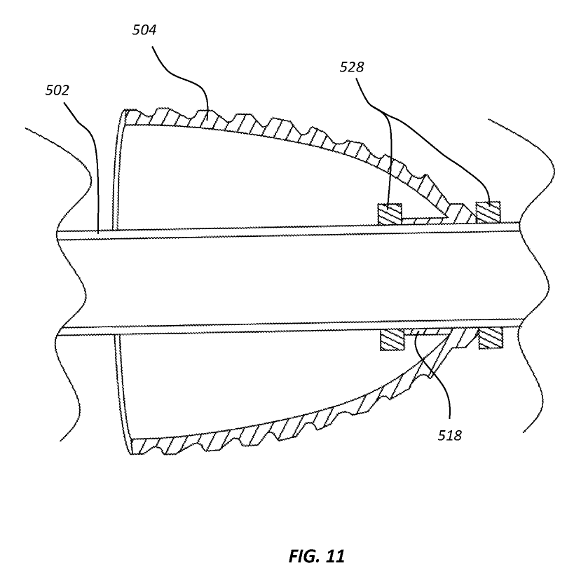

FIGS. 10 through 12 illustrate various alternative embodiments of locking mechanisms which may be utilized to secure the longitudinal position of the pressure seal relative to the proximal section of the catheter.

FIG. 10 illustrates a cross-sectional view of a pressure seal 304 positioned upon a catheter 302. The illustrated embodiment shows an example of a shaft collar type locking mechanism 328. The locking mechanism 328 is disposed about the conduit 318 of the pressure seal 304 and has an adjustable diameter to provide selective clamping of the conduit 318 to the catheter 302. When the locking mechanism 328 is in an open, larger diameter configuration, a caretaker may translate the pressure seal 304 upon the catheter 302 to a desired position. The caretaker may then adjust the locking mechanism 328 to a closed, smaller diameter configuration to effectively clamp the conduit 318 and catheter together 302, thereby locking the position of the pressure seal 304. The shaft collar type locking mechanism 328 may be formed as a hose clamp structure, circular spring clamp, quick-release clamp, or other adjustable diameter circular fastening devices known in the art.

The illustrated embodiment shows the locking mechanism 328 positioned near the center of the conduit 318. In other embodiments, the locking mechanism 328 may be positioned more distally or proximally. Further, although the illustrated embodiment includes a single locking mechanism 328, it will be understood that one or more additional locking mechanisms may also be included to provide a desired degree of locking of the pressure seal 304.

FIG. 11 illustrates another alternative pressure seal 504 which includes an example of a corkscrew or threaded engagement type locking mechanism 528. The illustrated pressure seal 504 includes a conduit 518 of relatively shorter length, which does not extend proximally beyond the outer member of the pressure seal 504. However, it will be understood that the same type of locking mechanism 528 may be applied to other embodiments having differently sized conduits. In the illustrated embodiment, the locking mechanism 528 is formed as a pair of threaded nuts or other suitable structures disposed on either side of the conduit 518.

The pressure seal 504 may be moved along the catheter 502 by rotating the locking mechanism structures about the catheter 502 to move them along corresponding threaded portions of the catheter, thereby also moving the pressure seal 504 to a desired position. Other embodiments may omit threaded engagement between the locking mechanism structures 528 and the catheter 502. For example, one or more of the locking mechanism structures 528 may be formed as an adjustable diameter clamp or shaft collar, such as described in relation to FIG. 10.

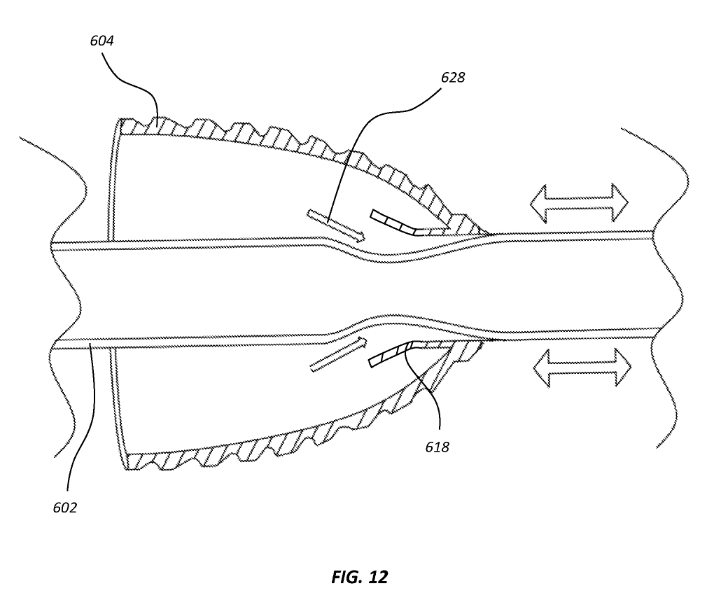

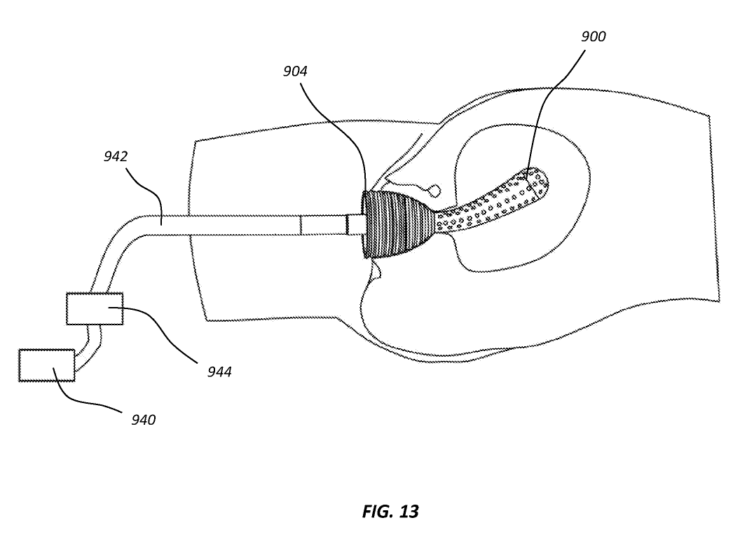

FIG. 12 illustrates another exemplary embodiment of a pressure seal 604 and catheter 602 showing an example of a friction fitting locking mechanism. In this embodiment, the inner diameter of the conduit 618 and the outer diameter of the catheter 602 are sized so as to fit tightly against each other. The resulting friction fit may be sufficient to prevent unintended translation of the pressure seal 604 upon the catheter 602. As shown by arrows 628, the friction fit may be overcome by pinching/collapsing a portion of the catheter 602 and/or stretching/expanding the conduit 618 to break the friction fit and allow desired longitudinal translation of the pressure seal 604.

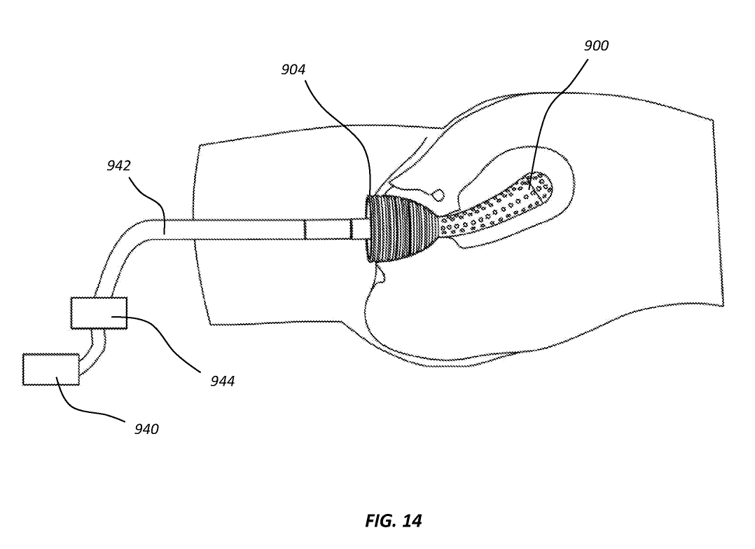

FIGS. 13 and 14 illustrate an exemplary use of a uterine contraction device 900 to assist in contracting a postpartum uterus. Although the illustrated uterine contraction device 900 includes a curved distal catheter section, it will be understood that any of the non-curved embodiments described herein may also be utilized as described. As shown in FIG. 13, the uterine contraction device 900 is coupled to a vacuum device 940 via a vacuum hose 942. The vacuum device may be electrically powered or manually powered, and may include any suitable device or plurality of devices capable of generating negative pressure as known in the art. In some embodiments, the vacuum device 940 may include a simple hand-powered pump. In use, negative pressure may be applied at levels suitable to assist in contracting the uterus without causing or aggravating injury to the uterus and surrounding anatomy.

The illustrated system also includes a filtering receptacle 944, which may be part of the vacuum device 940 or which may be a separate and distinct component. The filtering receptacle 944 may include one or more plastic flaps or other filtering components as known in the art for limiting fluid and/or tissue detritus travel past the receptacle 944 and into the filtering device 940. The filtering receptacle 944 is preferably interchangeable and readily disengaged from the vacuum system (e.g., via a button release or other suitable connection/detaching mechanism).

Once the uterine contraction device is properly positioned within a patient, as shown in FIG. 13, the vacuum device 940 may be actuated to generate negative pressure. The negative pressure is transmitted to the distal end of the uterine contraction device 900, where it reaches the uterus to assist in contracting the uterus. The pressure seal 904 remains in place within the vaginal canal to prevent pressure equilibrium between the uterine environment and the external environment. As shown in FIG. 14, after the negative pressure is applied for a suitable treatment time, the treated uterus may show a relatively more reduced and contracted state, which beneficially aids in reducing or preventing postpartum hemorrhaging.

The terms “approximately,” “about,” and “substantially” as used herein represent an amount or condition close to the stated amount or condition that still performs a desired function or achieves a desired result. For example, the terms “approximately,” “about,” and “substantially” may refer to an amount or condition that deviates by less than 10%, or by less than 5%, or by less than 1%, or by less than 0.1%, or by less than 0.01% from a stated amount or condition.

Elements described in relation to any embodiment depicted and/or described herein may be combinable with elements described in relation to any other embodiment depicted and/or described herein. For example, any element or component described in relation to a uterine contraction device of FIGS. 5 through 7 may be combinable with any element described in relation to a uterine contraction device of FIGS. 1 through 4. Likewise, any of the exemplary catheter elements, pressure seal configurations, locking mechanisms, or other components described in FIGS. 8 through 13 may be combined with or substituted for like components of the uterine contraction devices illustrated in FIGS. 1 through 4 or in FIGS. 5 through 7.

1. A medical device configured for insertion into the uterus to aid in contracting the uterus, the device comprising:
a catheter having a proximal end, a distal end, and a wall defining a lumen, the catheter being configured such that when inserted within a patient, the proximal end remains outside of the patient, and at least a portion of a distal section extends into the uterus,
wherein at least a portion of the distal section includes a plurality of perforations extending through the catheter wall to expose the lumen of the catheter, and
wherein the proximal end is attachable to a vacuum device to enable transmission of negative pressure through the lumen to the distal section; and
a pressure seal coupled to the catheter such that when the catheter is inserted into the patient, the pressure seal is disposed within the vaginal canal, the pressure seal having a bell shape that opens towards the proximal end of the catheter.

2. The device of claim 1, wherein the pressure seal is adjustably coupled to the catheter such that the pressure seal is translatable along a longitudinal axis of the catheter.

3. The device of claim 2, further comprising a locking mechanism to lock a selected position of the pressure seal relative to the catheter.

4. The device of claim 3, wherein the locking mechanism includes one or more grooves disposed on the catheter and one or more corresponding extensions disposed within a conduit of the pressure seal, the one or more extensions being configured to engage with the one or more grooves to lock relative position of the pressure seal upon the catheter.

5. The device of claim 3, wherein the locking mechanism includes a keyway disposed on the catheter and one or more corresponding keys disposed within a conduit of the pressure seal, the keyway including a plurality of offset slots for receiving the one or more keys upon rotation of the pressure seal to move the keys from the keyway into the offset slots.

6. The device of claim 3, wherein the locking mechanism includes friction fitting of the pressure seal and the catheter.

7. The device of claim 1, wherein the pressure seal includes a plurality of ridges disposed upon an outer surface of the pressure seal.

8. The device of claim 6, wherein the ridges are circumferentially oriented about the outer surface of the pressure seal.

9. The device of claim 1, wherein the catheter includes a proximal section formed separately from the distal section, the proximal section and the distal section being attached to form the catheter.

10. The device of claim 9, wherein the proximal section is formed from a material having a higher modulus of elasticity than the distal section.

11. The device of claim 10, wherein the pressure seal is formed from a material having a modulus of elasticity intermediate between that of the distal section and that of the proximal section.

12. A medical device configured for insertion into the uterus to aid in contracting the uterus, the device comprising:
a catheter having a proximal end, a distal end, and a wall defining a lumen, the catheter being configured such that when inserted within a patient, the proximal end remains outside of the patient, and at least a portion of a distal section extends into the uterus,
wherein at least a portion of the distal section includes a plurality of perforations extending through the catheter wall to expose the lumen of the catheter, and
wherein the proximal end is attachable to a vacuum device to enable transmission of negative pressure through the lumen to the distal section; and
a pressure seal adjustably coupled to the proximal section of the catheter such that the pressure seal is translatable along a longitudinal axis of the catheter.

13. The device of claim 12, further comprising a locking mechanism to lock a selected position of the pressure seal relative to the catheter.

14. The device of claim 13, wherein the locking mechanism includes one or more grooves disposed on the catheter and one or more corresponding extensions disposed within a conduit of the pressure seal, the one or more extensions being configured to engage with the one or more grooves to lock relative position of the pressure seal upon the catheter.

15. The device of claim 13, wherein the locking mechanism includes friction fitting of the pressure seal and the catheter.

16. The device of claim 12, wherein the pressure seal includes a plurality of ridges circumferentially oriented about an outer surface of the pressure seal.

17. The device of claim 12, wherein the catheter includes a proximal section formed separately from the distal section, the proximal section and the distal section being attached to form the catheter.

18. A method of treating postpartum hemorrhaging in a patient, the method comprising:
providing a medical device, the medical device including:
a catheter having a proximal end, a distal end, a distal section adjacent to the distal end, and a wall defining a lumen, the catheter being configured such that when inserted within a patient, the proximal end remains outside of the patient, and the distal section extends into the uterus, wherein at least a portion of the distal section includes a plurality of perforations extending through the catheter wall to expose the lumen of the catheter, and wherein the proximal end is attachable to a vacuum device to enable transmission of negative pressure through the lumen to the distal section; and
a pressure seal adjustably coupled to the proximal section of the catheter such that the pressure seal is translatable along a longitudinal axis of the catheter;
inserting the distal section of the medical device into the patient's uterus;
positioning the pressure seal at the patient's vaginal introitus; and
applying vacuum pressure to aid in contracting the uterus.

19. The method of claim 18, wherein the pressure seal has a bell shape that opens towards the proximal end of the catheter.

20. The method of claim 18, wherein the medical device further includes a locking mechanism to lock a selected position of the pressure seal relative to the catheter, the method further comprising locking the pressure seal in position prior to applying vacuum pressure.
